(19) United States
(12) Patent Application Publication
(10) Pub. No.: US 2024/0038390 A1
(43) Pub. Date: Feb. 1, 2024

(54) SYSTEM AND METHOD FOR ARTIFICIAL INTELLIGENCE BADED MEDICAL DIAGNOSIS OF HEALTH CONDITIONS

(71) Applicant: NEUROSPRING, INC., Napa, CA (US)

(72) Inventor: Mark BORSODY, Moraga, CA (US)

(21) Appl. No.: 18/256,063

(22) PCT Filed: Dec. 9, 2021
(86) PCT No.: PCT/US2021/062716

§ 371 Date: Jun. 6, 2023

Related U.S. Application Data
(60) Provisional application No. 63/123,179, filed on Dec. 9, 2020.

(51) Int. Cl. G16H 50/20; G16H 50/70

(52) U.S. Cl. CPC G16H 50/20; G16H 50/70

(57) ABSTRACT

Methods and medical devices comprising a processor comprising a plurality of data analytic process modules and a diagnostic integrator; a memory communicably coupled to the processor; an input/output device communicably coupled to the processor, the processor being configured to execute instructions stored in the memory to: cause the patent interface to record a first data from a subject; analyze the first data with a first of the plurality of data analytic process modules and determine a first diagnostic output; analyze the first date with a second of the plurality of data analytic process modules and determine a second diagnostic output; integrate the diagnostic outputs from the plurality of data analytic process modules and determine a unified final diagnosis to a subject.

### CROSS REFERENCE TO RELATED APPLICATIONS/PRIORITY

The present invention claims priority to U.S. Provisional Patent Application No. 63/123,179 filed Dec. 9, 2020, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### TECHNICAL FIELD

The present disclosure relates, generally, to the field of medical health and more specifically to artificial intelligence-based medical diagnosis of health conditions of a subject.

### BACKGROUND

Artificial intelligence has become a disruptive technology in healthcare industry with the potential to transform patient care as well as administrative processes. Artificial intelligence-based systems reduce the diagnostic workload for physicians, most of whom are overworked to the point of complete exhaustion. Additionally, these systems tend to bring down the rates of wrong diagnosis. However, the existing artificial intelligence-based systems are not completely accurate and lack early detection and diagnosis of some diseases. Also, the existing systems require the involvement of a physician for confirmation of the diagnosed medical health condition.

Therefore, there is a need for an improved and accurate artificial intelligence-based system that overcomes the above-stated disadvantages.

### SUMMARY OF THE INVENTION

Wherefore, it is an object of one or more embodiments of the presently disclosed invention to overcome the one or more or all of the above-mentioned shortcomings and drawbacks associated with the current technology.

The presently disclosed invention relates to methods and medical devices comprising, a processor comprising a plurality of data analytic process modules and a diagnostic integrator; a memory communicably coupled to the processor; an input/output device communicably coupled to the processor, the processor being configured to execute instructions stored in the memory to: cause the patent interface to record a first data from a subject; analyze the first data with a first of the plurality of data analytic process modules and determine a first diagnostic output; analyze the first date with a second of the plurality of data analytic process modules and determine a second diagnostic output; integrate the diagnostic outputs from the plurality of data analytic process modules and determine a unified final diagnosis to a subject. According to a further embodiment the input/output device includes at least one sensor. According to a further embodiment the at least one sensor includes a video camera and a microphone. According to a further embodiment the at least one sensor further include one or more of and thermal camera, thermometer, electrocardiography sensor, photoplethysmography sensor, electromagnetic pulse monitor, accelerometer, and a gyroscope. According to a further embodiment the input/output device includes one or more of a speaker and video display screen. According to a further embodiment the input/output device comprises a headset wearable by the subject. According to a further embodiment the headset comprises one or more external cameras facing in a direction not towards a face of the subject when the subject is wearing the headset, one or more internal cameras facing toward the face of the subject when the subject is wearing the headset, one or more speakers, a semi-transparent augmented reality visor, and one or more microphones oriented proximate to a mouth of the of the subject when the subject is wearing the headset, one or more speakers oriented proximate to ears of the subject. According to a further embodiment the input/output device comprises one or more stimulators positioned to deliver sensory stimulation to face, scalp, and/or other body part of the subject, wherein the stimulation delivered is on or more of thermal, vibratory, tactile, and/or electrical in nature. According to a further embodiment the input/output device comprises one or more peripherals positioned on one or both ankles and/or one or both wrists of the subject, the peripherals including adhesive and or having a circular shape to remain frictionally attached to a subject wrapped around a limb of the patient, the peripherals including one or more sensors and or one or more stimulators. According to a further embodiment, the medical device further comprises a plurality of fixed equipment, wherein the each of the plurality fixed equipment is fixed to a respective one of a vehicle, a building, a medical transport, and a furniture. According to a further embodiment a first equipment of the plurality of fixed equipment is fixed to an ambulance and includes a third person video camera, a video console, one or more speakers, and a microphone. According to a further embodiment a second equipment of the plurality of fixed equipment is fixed to a medical transport used to move a patient in and out of the ambulance vehicle. According to a further embodiment the processor is further configured to cause the input/output device to display graphic and/or other visual information to the subject in response to verbal response received from subject to auditory, the subject verbal response being in response to visual or auditory output from the medical device. According to a further embodiment the plurality of data analytic process modules includes at least two of includes a machine learning process module, a syndrome analyzer module, a case matching module, and a diagnostic code linking module. According to a further embodiment the processor is further configured to convert patient speech to text and cause speakers to auditorily respond to patient with spoken text. According to a further embodiment the processor is further configured to access one or more databases. According to a further embodiment the machine learning process module determines likelihood of proper diagnosis of a given disease or condition in the subject based on a combined association of a plurality of data inputs and the incidence of given disease or condition, where the data inputs are collected from the subject through the input/output device, and data inputs include one or more of presence of sudden numbness or weakness in body of the subject, a National Institutes of Health Stroke Scale (NIHSS) score, indication of tobacco, an age, a race, a sex, indication of dyslipidemia, indication of atrial fibrillation, indication of high blood pressure, current systolic blood pressure, current diastolic blood pressure, current glucose level, medications the subject is currently taking, indication of subject family history of stroke, indication of coronary artery disease, and current heart rate. According to a further embodiment syndrome analyzer module determines likelihood of proper diagnosis of a given disease or condition in the subject based on a presence or absence of one or more data elements, where data elements are symptoms associated with the disease or condition. According to a further embodiment, the medical device further comprises a therapy deliverer, wherein, after the processor determines a diagnosis of a disease, the processor is further configured to case the therapy deliverer to deliver a therapy directly to the subject. According to a further embodiment the therapy deliverer delivers one of injection of medication and electrical nerve stimulation to the subject.

The present disclosure relates, generally, to the field of medical health and more specifically to artificial intelligence-based medical diagnosis of health conditions of a subject.

Embodiments of the disclosed invention are related to an artificial intelligence-based medical diagnostic system (hereinafter AID system) for diagnosing health condition of a subject and directing refined treatment to the subject based on the diagnosed health condition. The AID system extracts data inputs associated with the subject through one or more sensors associated with the AID system during one or more evaluation of the subject, and potentially from other sources of information related to the subject.

Embodiments of the disclosed invention are related to the AID system that evaluates a speech signal from the subject with facilitation of a plurality of spectral analytics processes. Each of the plurality of spectral analytics processes is configured for diagnosing qualitative abnormalities in a parallel manner. The evaluation of the speech signal using the plurality of spectral analytics processes is done to obtain an output. The output is associated with quality of speech and corresponds to a determination of abnormal or normal speech quality and/or to the type of normality of speech quality.

Embodiments of the disclosed invention are related to the AID system that ensures accurate identification of normalities and abnormalities. A plurality of computer vision processing capabilities may be employed by the AID system in a substantially parallel manner to examine a video or any visual representation of the subject and/or the subject's environment.

Embodiments of the disclosed invention are related to the AID system that utilizes a plurality of data analytic process modules. The plurality of data analytic process modules includes a machine learning process module, a syndrome analyzer module, a case matching module, and a diagnostic code linking module. The machine learning process module (MLP) analyzes the data inputs extracted from the user or from third party platforms (as additional potential sources of medical history and physical examination findings) by mapping to pre-established diagnoses present in one or more database. The data inputs serve as features into which different data elements provided by the subject must, in a preferred embodiment, fit.

Embodiments of the disclosed invention are related to the AID system that generates one or more keywords and phrases as a part of diagnostic evaluation of the subject. The one or more keywords and phrases are linkable to healthcare service billing records. The healthcare service billing records contains a final diagnosis provided by a treating physician. The healthcare billing records may include or reference the International Classification of Diseases or other such index as a means to standardize terminology and diagnosis. By identifying the one or more keywords and phrases from a large population of the subject medical records and linking them to the diagnoses in the healthcare billing records, the one or more keywords and phrases may be used as indicators of an individual subject's diagnosis during that subject's evaluation.

Embodiments of the disclosed invention are related to the AID system in which the data inputs and data elements are analyzed in different manners by the plurality of data analytic process modules to diagnose health condition of the subject. The AID system may utilize more than one data analytic process of the plurality of data analytic process modules for analysis of the data inputs and the data elements at one time.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

FIG. 1 illustrates a block diagram of an artificial intelligence-based medical diagnostic (AID) system, in accordance with an embodiment of the presently disclosed invention;

FIG. 2 illustrates a block diagram of different types of data sources used in the MLP module and storing of the data inputs in a database of the AID system, in accordance with an embodiment of the presently disclosed invention;

FIG. 3 illustrates a schematic diagram of a two-dimensional diagnostic process utilized by the AID system, in accordance with an embodiment of the presently disclosed invention;

FIG. 4 illustrates a schematic diagram of combined outputs obtained from the two-dimensional diagnostic process shown in FIG. 3;

FIG. 5 illustrates a schematic diagram of a three-dimensional diagnostic process utilized by the AID system shown in FIG. 3, in accordance with an embodiment of the presently disclosed invention;

FIG. 6 illustrates a working example of a view of an input/output device associated with the AID system, in accordance with an embodiment of the presently disclosed invention;

FIG. 7 illustrates visualization of vantage points captured by the input/output device associated with the AID system, in accordance with an embodiment of the presently disclosed invention;

FIG. 8A illustrates an ambulance configured with one or more sensors associated with the AID system, in accordance with an embodiment of the presently disclosed invention;

FIG. 8B illustrates a schematic diagram of internal and external view of the ambulance configured with the one or more sensors, in accordance with an embodiment of the presently disclosed invention;

FIG. 9 illustrates a flow diagram of a method for diagnosing health condition of a subject and directing refined treatment to the subject based on the diagnosed health condition, in accordance with an embodiment of the presently disclosed invention;

FIG. 10 illustrates a flowchart for functioning of the AID system, in accordance with an embodiment of the presently disclosed invention;

FIG. 11 illustrates steps of an embodiment for detecting symptoms relevant to neurological emergency within a term hierarchy using the AID system of FIG. 1, in accordance with an embodiment of the presently disclosed invention;

FIG. 12 illustrates steps of an embodiment with a process flowchart for asking questions to a patient related to symptoms of dizziness using the AID system of FIG. 1, in accordance with an embodiment of the presently disclosed invention;

FIG. 13 illustrates steps of an embodiment for detecting symptoms relevant to abnormal vision using the AID system of FIG. 1, in accordance with an embodiment of the presently disclosed invention; and

FIG. 14 illustrates steps of an embodiment with a process flowchart for asking questions to a patient related to symptoms of abnormal vision, using the AID system of FIG. 1, in accordance with an embodiment of the presently disclosed invention.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures, and are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. Where spatial directions are given, for example above, below, top, and bottom, such directions refer to the artificial intelligence-based medical diagnostic system as represented in whichever figure is currently described, unless identified otherwise.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “substantially” means that the property is within 80% of its desired value. In other embodiments, “substantially” means that the property is within 90% of its desired value. In other embodiments, “substantially” means that the property is within 95% of its desired value. In other embodiments, “substantially” means that the property is within 99% of its desired value. For example, the term “substantially complete” means that a process is at least 80% complete, for example. In other embodiments, the term “substantially complete” means that a process is at least 90% complete, for example. In other embodiments, the term “substantially complete” means that a process is at least 95% complete, for example. In other embodiments, the term “substantially complete” means that a process is at least 99% complete, for example.

The term “substantially” includes a value within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

The term “about” includes a value within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

In addition, the invention does not require that all the advantageous features and all the advantages of any of the embodiments need to be incorporated into every embodiment of the invention.

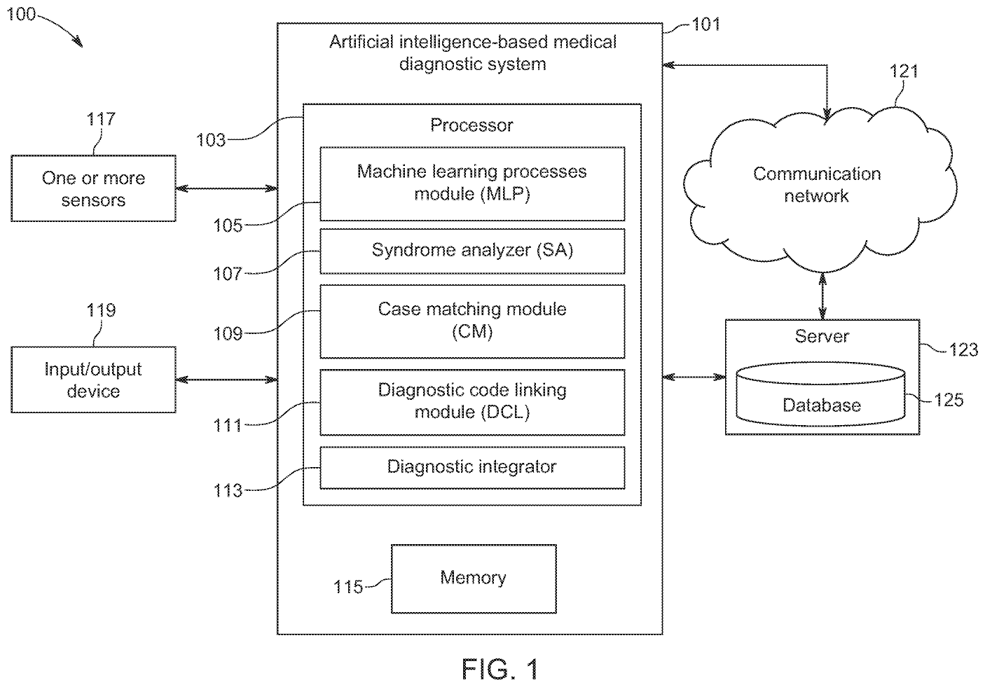

Turning now to FIGS. 1-14, a brief description concerning the various components of the present invention will be briefly discussed.

Reference will be made to the figures, showing various embodiments of an artificial intelligence-based medical diagnostic (hereafter AID) system for diagnosing subject's health condition and directing refined treatment to the subject based on the diagnosed health condition.

The present invention discloses an AID system for diagnosing a subject's health condition and directing refined treatment to the subject based on the diagnosed health condition. Diagnosis of the subject's health condition is performed using multidimensional analytic processes. The AID system automatically directs or delivers therapeutic intervention/treatment through additional or integrated components of the AID system.

Referring to FIG. 1, a block diagram 100 of an AID system 101 is shown. The AID system 101 comprises a processor 103 and a memory 115. The processor 103 comprises one or more modules, such as: a machine learning processes module (hereinafter MLP) 105, a syndrome analyzer module (SA) 107, a case matching module (CM) 109, a diagnostic code linking module (DCL) 111, and a diagnostic integrator 113. In addition, the AID system 101 is connected to one or more sensors 117 and an input/output device 119. Further, the AID system 101 is connected with a communication network 121, which is configured to communicatively couple the AID system 101 to a server 123 and a database 125.

The AID system 101 extracts data inputs associated with the subject. The subject may be a patient whose medical health condition needs to be diagnosed. The subject may be any individual who needs medical assistance. The subject may be any individual who wants to keep a track of their medical health condition. The AID system 101 extracts the data inputs through at least one of the subject, a physician, other healthcare providers, other individuals familiar with the subject or familiar with events related to the subject, or any third-party data repository. The data inputs extracted by the AID system 101 may correspond to clinical and non-clinical information. In some embodiments, the data inputs include, but are not necessarily limited to, data associated with the medical history of the subject, the subject's family medical record, explanation of any health-related symptoms that the subject is having, medication the subject uses, the subject's allergies, subject physical examination findings, and basic laboratory testing results on the subject.

The medical history and family medical record of the subject may be extracted from the subject through the subject's interaction with the AID system 101, from other people with knowledge of the subject or the events affecting the subject, and/or through any third-party platform that stores past medical record. The data associated with explanation of any health-related symptoms that the subject is having may also be extracted from the subject interaction with the AID system 101. The subject interacts with the AID system 101 via the input/output device 119 associated with the AID system 101 and the subject. The AID system 101 may collect speech content of the subject while the subject is interacting with the AID system 101. The AID system 101 may ensure accurate interpretation of the speech content of the subject to identify symptoms of diseases or any health condition. The AID system 101 may analyze the speech content of the subject and extracts data from the speech content of the subject by using one natural language processing platform or a plurality of natural language processing platforms in a substantially parallel manner. Data contained therein is then identified in the speech content of the subject by each individual natural language processing platform of the plurality of natural language processing platforms.

In addition, the AID system 101 determines identity and/or nature of said data by a predefined means. In some embodiments of the invention, the predefined determination is a based on a simple consensus or majority parameter associated with the plurality of natural language processing platforms. In one example, each natural language processing platform of the plurality of natural language processing platforms may be considered equally capable of determining identity and/or nature of said data. In general, natural language processing is a collective term referring to automatic computational processing of human languages during interactions between computers and humans. In another example, certain natural language processing capabilities are preferentially selected or otherwise weighted to determine presence and/or nature of data contained in the speech content of the subject based on the design, training, or accuracy of said natural language processing capability. In examples of specific embodiments, one or more natural language processors are trained solely to recognize slang or jargon terminology.

In some embodiments of the present invention, the AID system 101 evaluates a speech signal from the subject with facilitation of a plurality of spectral analytics. Each of the plurality of spectral analytics diagnoses qualitative abnormalities in a parallel manner. The evaluation of the speech signal using the plurality of spectral analytics is done to obtain an output. The output is associated with quality of speech. The output corresponds to a single diagnostic determination of abnormal or normal speech quality (dysarthria).

The AID system 101 performs data extraction from the speech signal and the speech content of the subject. Additionally, data associated with physical examination findings may be extracted through computer vision analytics that assess various aspects of physical condition of the subject. The various aspects of physical condition of the subject include but may not be limited to weakness in face or limbs of the subject, expressions on face of the subject, sleepy eyes, shivering in body of the subject, the condition of the subject's skin or clothing, and/or the objects found in the subject's immediate surroundings. The AID system 101 extracts the data associated with physical examination findings through computer vision analytics using the one or more sensors 117. The one or more sensors 117 with facilitation of computer vision analytics capture one or more images focusing on abnormalities of, and around, the subject. The AID system 101 ensures accurate identification of normalities and abnormalities. A plurality of computer vision processing capabilities may be employed in a substantially parallel manner to examine a video or any visual representation of the subject. In some embodiments of the invention, the identification of normalities or abnormalities is a simple consensus or majority of the plurality of computer vision processing capabilities. Each of the computer vision processing capabilities is considered equally capable of identifying normalities and abnormalities. In an exemplary embodiment of the invention, certain computer vision capabilities of the plurality of computer vision processing capabilities are preferentially selected or otherwise weighted to identify normalities and abnormalities contained in the one or more images of the based on the design, training, or accuracy of said computer vision capability.

The AID system 101 is connected with the communication network 121. The communication network 121 provides a medium to the AID system 101 to connect to the server 123 and the database 125. In one embodiment of the present invention, the communication network 121 is internet. In another embodiment of the present invention, the communication network 121 is a wireless mobile network. In yet another embodiment of the present invention, the communication network 121 is a combination of the wireless and wired network for optimum throughput of data extraction and transmission. The communication network 121 includes a set of channels. Each channel of the set of channels supports a finite bandwidth. The finite bandwidth of each channel of the set of channels is based on capacity of the communication network 121. The communication network 121 connects the AID system 101 to the server 123 and the database 125 using a plurality of methods. The plurality of methods used to provide network connectivity to the AID system 101 may include 2G, 3G, 4G, 5G, and the like.

The AID system 101 is communicatively connected with the server 123. In general, server is a computer program or device that provides functionality for other programs or devices. The server 123 provides various functionalities such as sharing data or resources among multiple clients or performing computation for a client. However, those skilled in the art would appreciate that the AID system 101 may be connected to a greater number of servers. Furthermore, it may be noted that the server 123 includes the database 125.

The server 123 handles each operation and task performed by the AID system 101. The server 123 stores one or more instructions for performing the various operations of the AID system 101. In one embodiment, the server 123 is located remotely. The server 123 is associated with an administrator. In addition, the administrator manages the different components associated with the AID system 101. The administrator is any person or individual who monitors the working of the AID system 101 and the server 123 in real-time. The administrator monitors the working of the AID system 101 and the server 123 through a communication device. The communication device includes laptop, desktop computer, tablet, a personal digital assistant, and the like. In addition, the database 125 stores the data inputs associated with the subject. The database 125 organizes the data inputs using model such as relational models or hierarchical models. The database 125 also stores data provided by the administrator.

The AID system 101 comprises the memory 115. The memory 115 comprises at least one of RAM, ROM, EEPROM, flash memory or other memory technology, CD-ROM, digital versatile disks (DVD) or other optical disk storage, magnetic cassettes, magnetic tape, magnetic disk storage, or any other storage medium which can be used to store the desired information, and which can be accessed by the AID system 101. The memory 115 may include non-transitory computer-storage media in the form of volatile and/or nonvolatile memory. The memory 115 may be removable, non-removable, or a combination thereof. Exemplary memory devices include solid-state memory, hard drives, optical-disc drives, and the like.

The AID system 101 utilizes a plurality of data analytic process modules of the processor 103. The plurality of data analytic process modules of the processor 103 includes the MLP 105, the SA 107, the CM 109, and the DCL 111 modules. The MLP 105 analyzes the data inputs extracted from the subject evaluation(s) by mapping to pre-established diagnosis present in the database 125. In some embodiments, the data inputs needed for the MLP 105 must match with the database 125 that serve to train the MLP 105. The data inputs serve as features into which different data elements obtained during the subject evaluation(s) by the subject preferably must fit. In some embodiments, different portions of the MLP 105 may be engaged or otherwise be used in the diagnostic evaluation in a manner determined by the data elements derived from the data inputs the subject evaluation(s) reveal to the AID system 101.

The data elements provided by the subject evaluation(s) may fit into definitions of classic syndromes that are linked to specific diagnosis. The definitions of classic syndromes are provided in the medical literature. The term ‘syndrome’ as used here and in common conversation includes not only symptoms but other medical history, physical examination findings, and diagnostic testing results as well. In some embodiments of the invention, it is not necessary to have all parts of the definitions of a syndrome satisfied with the data elements provided by the subject evaluation(s), nor do all of the data elements provided by the subject evaluation(s) have to be represented in or accounted for by the definitions of a classic syndrome, for the subject to be diagnosed with a given syndrome by the SA 107. The degree to which a syndrome's definitional elements must be satisfied can be predetermined, can vary between different syndromes, and can be determined on a syndrome-by-syndrome basis.

The CM 109 performs mapping of the data elements provided by the subject evaluation(s) with the database 125. The data elements and/or narrative story provided by the subject are compared against summary records from other subjects/patients who have established diagnoses, as recorded in the medical literature and/or documented in electronic medical records or databases. In general, lexical, semantic, and/or other similarities may be used in the comparison process. In addition, multiple matching records may be rank ordered, weighted based on the degree of similarity or dissimilarity, counted as number of similar/dissimilar records, or otherwise quantified to establish a measure of confidence to relate the established diagnosis to the subject under evaluation. Further, content-based filtering, collaborative-based filtering, recommendation engines, and other means may be used to establish the measure of confidence, employing similarity, and distance, or other metrics in the analysis. Any number of the subject features can be employed for case matching process with predetermined requirements set by the AID system 101 for the number of data elements that are then required to be matched between the data inputs provided by the subject evaluation(s) and the data inputs present in the database 125.

The processor 103 includes the DCL 111. The AID system 101 generates or otherwise identifies one or more keywords and phrases as a part of diagnostic evaluation of the subject. The one or more keywords and phrases are linkable to healthcare service billing records. The healthcare service billing records contains a final diagnosis provided by treating physician. The healthcare service billing records may include the International Classification of Diseases or other such index as a means to standardize terminology and diagnosis. By identifying the one or more keywords and phrases from a large population of the subject medical records and linking them to the diagnoses in the healthcare service billing records, the one or more keywords and phrases may be used as indicators of an individual subject's diagnosis during that subject's evaluation(s). The measure of certainty of a keyword/phrase linked to a diagnosis obtained by the DCL module may be numerical, proportional, based upon frequency of occurrence, determined by specificity, and/or involve some other measure of the quality or strength of the link between the one or more keywords and phrases and the diagnostic code.

The data inputs and the data elements are analyzed in different manners by the plurality of data analytic process modules to diagnose health condition of the subject. The AID system 101 may utilize more than one data analytic process of the plurality of data analytic process modules of the processors 103 for analysis of the data inputs and the data elements at one time. In some embodiments, the AID system 101 utilizes combination of two data analytic process modules of the plurality of data analytic process modules. In some embodiments, the AID system 101 utilizes three or more data analytic process modules of the plurality of data analytic process modules at the same time to analyze the data inputs and the data elements. The AID system 101 uses the plurality of data analytic process modules simultaneously using the diagnostic integrator 113. The diagnostic integrator 113 integrates diagnostic outputs from the plurality of data analytic process modules to determine a unified final diagnosis to the subject and/or user of the AID system 101.

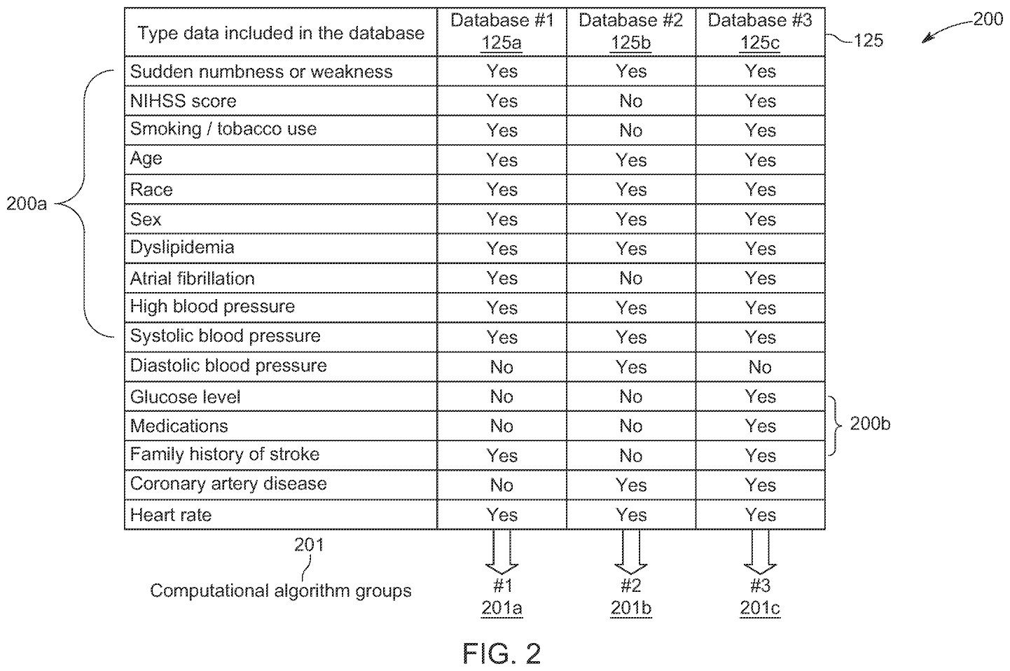

Referring to FIG. 2, a block diagram 200 illustrating a general overview of an embodiment of collection of the data sources used in MLP 105 to train and compile it in the database 125 of the AID system 101 is shown. Initial evaluation of the subject involves extraction or collection of a predefined number of data inputs 200a of the data inputs from the database 125. For example, in the illustration shown in FIG. 2, the predefined number is 10. However, any number may be suitably used to specify this predefined number, without deviating from the scope of the present disclosure. The 10 data inputs 200a includes data related to sudden numbness or weakness in body of the subject, National Institutes of Health Stroke Scale (NIHSS) score, use of tobacco, age, race, sex, dyslipidemia, atrial fibrillation, high blood pressure, and systolic blood pressure as examples. In one embodiment of the invention, the database 125 is composed of three separate databases: database #1 125a, database #2 125b, and database #3 125c that each had been employed to train separate groups of machine learning model (MLM) 201 representing a subset of the MLP module 105. The data inputs obtained during the subject evaluation(s) match those included in a certain database of the three databases. The specific MLM 201 is then engaged to diagnose health condition of the subject. In this example, the 10 data inputs 200a identified during a subject's evaluation match those recorded in database #1 125a, therefore, preferably only machine learning model of the group of MLM 201a which are trained on the database #1 125a are used for diagnostic analysis at that point of time. Additional MLM trained on databases that also contain the 10 data inputs 200a may also be employed for diagnostic purposes either routinely or depending upon the output provided by MLM #1 201a.

Continuing with this example: if additional information is subsequently collected about the subject in a follow-up evaluation, or is obtained from other sources, and additional data inputs are found in that information, a different or additional group(s) of MLM can be engaged in the diagnostic evaluation based upon the additional group(s) ability to handle the expanded number of the data inputs. The additional data inputs 200b are also extracted or collected by the AID system 201, expanding upon those previously collected data inputs 200a. In this example, the additional data inputs 200b include medication use, family medical history, and glucose level. The additional data inputs 200b of the data inputs are represented in database #3 125c that also contains the previously collected data inputs 200a. In this example, the MLM #3 201c which are trained on the database #3 125c would then be used for diagnostic analysis at that point of time, either to replace or to complement the initial diagnosis provided by the MLM #1 201a. In this example, the MLM #2 201b trained upon database #2 125b is preferably not used in either assessment of the subject/patient because the database does not contain the complete list of original data inputs 200a nor the supplementary data inputs 200b.

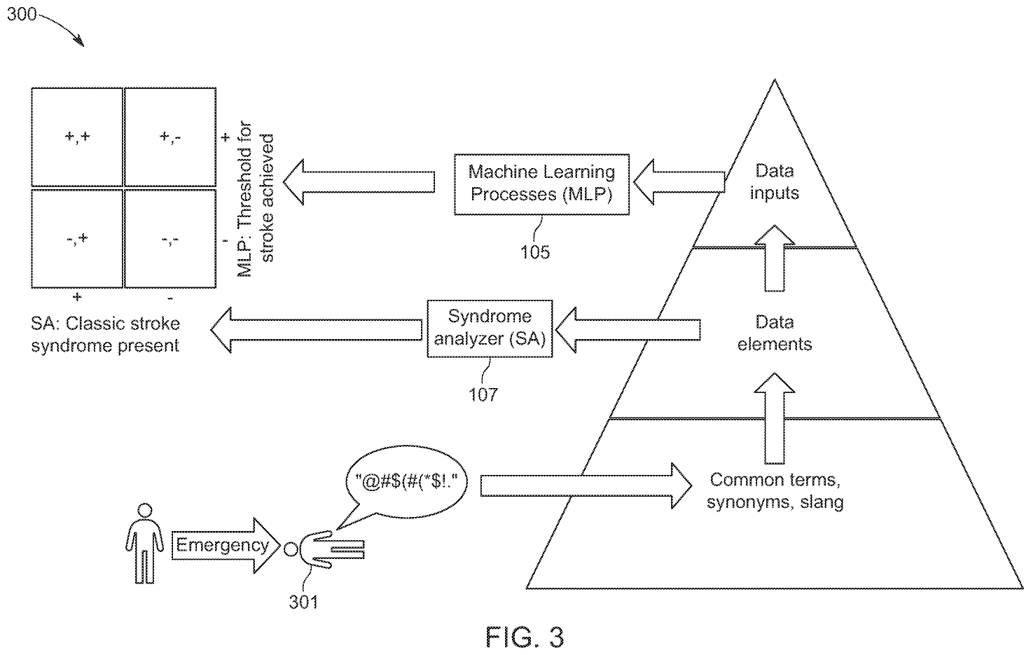

Referring to FIG. 3, a schematic diagram 300 illustrating two-dimensional diagnostic process utilized by the AID system 101 is shown. The two-dimensional diagnostic process correspond to simultaneous use of two data analytic process modules of the plurality of data analytic process modules. In an example, the two data analytic process modules of the plurality of data analytic process modules are the MLP 105 and the SA 107. In some embodiments, other combinations of two data analytic process modules of the plurality of data analytic process modules may be used. In this example, information provided by a subject evaluation(s) is converted to text after recognition of synonyms and slang terminology. The subject 301 is a patient whose health condition has to be diagnosed. The data elements are extracted from some portions of the converted text and are evaluated by the SA 107 to identify matching classic syndromes, some of which may be associated with or pathognomonic for a health condition. Some but not all the data elements also suffice as data inputs that are required for operation of the MLP 105, and not all data inputs for the MLPs must represent data elements for the SA. Sufficient completion of the MLP's data input requirements allows the MLP 105 to calculate a diagnostic probability of a certain diagnosis or otherwise provide a diagnosis. The distinct diagnostic outputs of the SA 107 and the MLP 105, which are based on the same speech utterances provided by the same subject 301, may then agree or disagree on the diagnosis of the health condition. In this situation, the diagnostic integrator 113 is employed to compare the diagnoses derived from the MLP 105 and the SA 107 for the purpose of determining a single, unified diagnosis to be provided to the subject 301 and/or healthcare providers. In this example, if both the MLP 105 and the SA 107 agree on the diagnosis of any particular health condition, e.g. stroke, then confidence in that diagnosis increases and the AID system 101 then triggers a certain course of action such as the administration of emergency treatment or the direction of transportation of the patient.

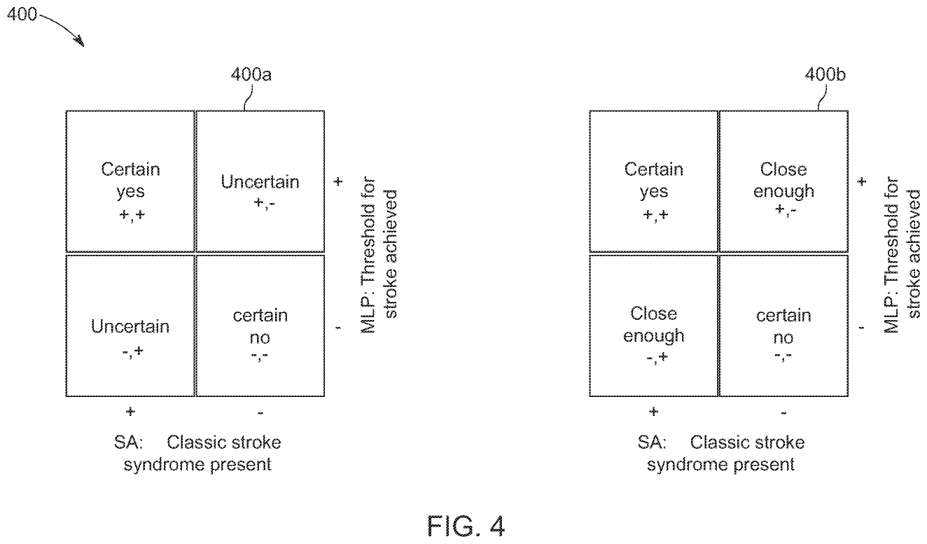

Referring to FIG. 4, a schematic diagram 400 giving an overview of examples of combined outputs (400a, 400b) obtained from two-dimensional diagnostic process is illustrated. Diagnosis of stroke is shown with a “+” (plus) sign. In addition, diagnosis of ‘not stroke’ or the diagnosis of another medical condition is shown with a “−” (minus) sign. As shown in 400a, the requirement for agreement in the diagnostic integrator 113 means the two data analytic process modules serve to ‘double check’ each other's diagnosis. Similarly, if both the MLP 105 and the SA 107 agree that the subject's 301 diagnosis is not stroke, or agree on the diagnosis of another, non-stroke condition, then confidence that the subject's 301 diagnosis is not stroke increases. If that other disease can be positively identified, the best course of action then leads to disease-specific treatment for that other disease. Disagreement between the two data analytic process modules (MLP and SA) creates an uncertain diagnosis, which may lead to actions such as a ‘second opinion’ evaluation by a human physician through a video conferencing connection, or routing of the subject 301 to a certain hospital with skilled staff available to evaluate the subject 301 in-person.

Alternately, as shown in 400b, for the example of an AID system that diagnoses stroke, the diagnostic integrator 113 may allow the diagnosis of any health condition such as stroke to be given to the subject and/or user if either or both of the two data analytic process modules of the plurality of data analytic process modules detected the health condition such as stroke. The diagnosis of any health condition such as stroke, detected by either or both of the two data analytic process modules, is done for purpose of not missing any subject with said health condition. In an example, the AID system 101 operates as an initial screening tool for stroke or any health condition within broader population of neurological emergencies for the purpose of immediately referring certain patients to a physician evaluation that then confirms the diagnosis.

In some embodiments, the diagnosis of stroke may be provided to the subject 301 in which both of the two data analytic process modules of the plurality of data analytic process modules agree on the diagnosis of stroke or in which either of the two data analytic process modules reaches the diagnosis of stroke, but in which a potentially dangerous medication would be administered or directed to the subject 301 only when both of the two data analytic process modules agree on the diagnosis. In addition, only safer treatments would be administered or directed to the subject 301 when only one of the two data analytic process modules reaches the diagnosis of stroke.

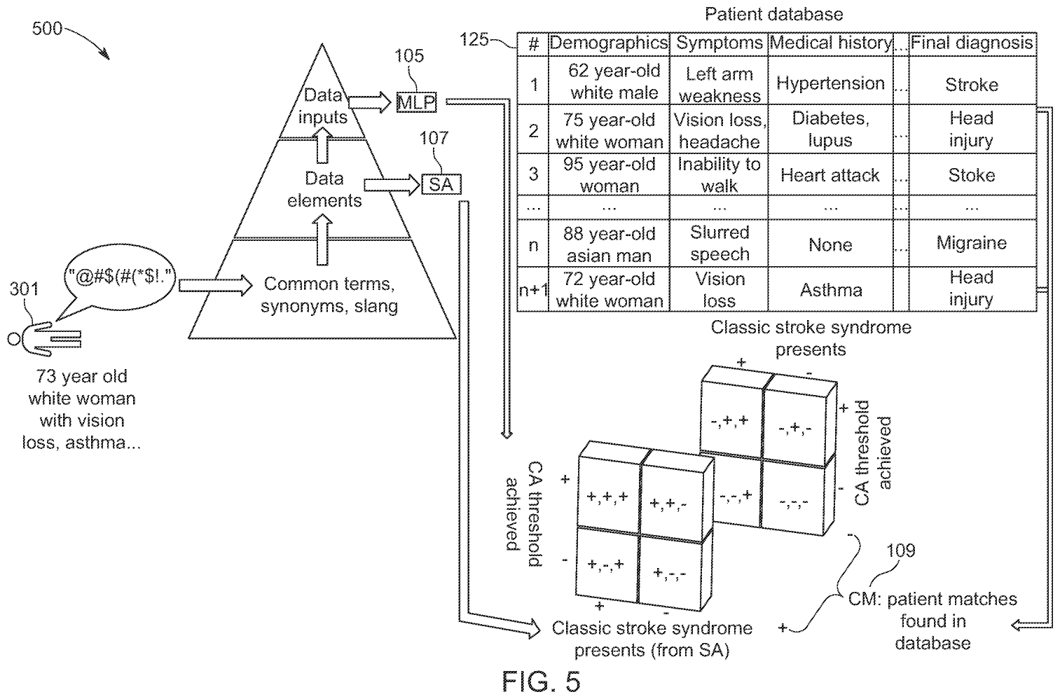

The diagnostic integrator 113 is not limited to use of two data analytic process modules of the plurality of data analytic process modules. More than two data analytic process modules of the plurality of data analytic process modules may be used by the diagnostic integrator 113 for additional, complementary dimensions for diagnostic confirmation. Referring to FIG. 5, a schematic diagram 500 of three-dimensional diagnostic process utilized by the AID system 101 is illustrated. The three-dimensional diagnostic process shown in the schematic diagram 500 corresponds to simultaneous use of three data analytic process modules of the plurality of data analytic process modules. In one embodiment, the AID system 101 employs three data analytic process modules of the plurality of data analytic process modules and an array of 2×2×2 is created within the diagnostic integrator 113. In an example, the AID system 101 employs the MLP 105, the SA 107, and the CM 109 modules. The subject 301 is under evaluation and is matched to the most similar patients from the pre-established or developing patient database 125. In this example, at least two of three data analytic process modules of the plurality of data analytic process modules must agree on a particular health condition for the subject 301 to be provided with a diagnosis such as stroke. If the subject 301 under evaluation is diagnosed with stroke by only one of the three data analytic process modules, the diagnosis is considered uncertain, and in some embodiments the uncertainty triggers evaluation by a human physician. In other embodiments, the achievement of an uncertain diagnosis triggers additional evaluation or re-evaluation of the subject by the AID system, additional diagnostic testing of the subject, and/or further searching of and for data sources related to the subject including additional human sources of information. In some embodiments, further searching of and for data sources by the AID system may take the form of accessing patient electronic medical records from healthcare systems in states and countries where the subject/patient currently and/or previously lived, and/or in identifying and contacting relatives after they are identified by social media searches.

The AID system 101 may employ or utilize any number of data analytic process modules which may similarly be coordinated into a multidimensional array. In an example, four data analytic process modules may be employed in a 2×2×2×2 array, and various combinations of results may be defined as necessary to establish or exclude certain diagnoses for the subject. Additionally, the diagnostic decisions produced by each of the plurality of data analytic process modules need not be considered equal by the diagnostic integrator 113. Weighting of certain diagnostic decisions derived by operationally superior data analytic process modules of the plurality of data analytic process modules may be employed. Operational superiority of any data analytic process of the plurality of data analytic process modules may be predetermined or else determined for a individual subject's diagnosis as a result of measures obtained during evaluation of the individual subject.

Failure of any data analytic process of the plurality of data analytic process modules designed to identify a specific neurological emergency, e.g. stroke, against the broader group of non-stroke conditions may not necessarily establish any specific non-stroke diagnosis, such as seizure or traumatic brain injury. In addition, specific data analytic process modules of the plurality of data analytic process modules may be needed for each medical emergency condition or disorder for the purpose of rendering a positive diagnosis for that condition or disorder. In an example, the AID system 101 may require a plurality of diagnostic integrators. Each of the plurality of diagnostic integrators corresponds to the diagnostic integrator 113. Each of the plurality of diagnostic integrators operating on two or more data analytic process modules of the plurality of data analytic process modules may be intended for the diagnosis of a specific medical condition. Accurate diagnosis of the subject 301 (or any patient) may then require that, for example, a stroke-specific diagnostic integrator confirm the diagnosis of stroke and the plurality of diagnostic integrators used for conditions other than stroke may confirm that the subject's diagnosis is none of the other conditions. To achieve this analysis, the plurality of diagnostic integrators may be needed in a hierarchy.

The AID system 101 may act to primarily diagnose neurological emergencies for the purpose of identifying certain medical conditions that may be immediately treated after diagnosis. In an example, one such condition is ischemic stroke. Certain new treatments for ischemic stroke may be directed to the subject 301 (any patient) by means of nerve stimulation. In general, ischemic stroke occurs when a blood clot blocks or narrows an artery leading to the brain. In an example, any one of facial nerve, vagus nerve, trigeminal nerve, or other cranial or peripheral nerves, dilate arteries of the brain, head, or neck of the subject 301. Dilation of the arteries leads to increases in blood flow to the brain (increased cerebral blood flow and perfusion). These nerves are paired, with one nerve on each side of the body, and the effect of the nerve stimulation is primarily ipsilateral. In general, ipsilateral refers to dilation of arteries and increase in blood flow to the brain that occurs on the same side as the stimulated nerve.

In an example, the AID system 101 determines the side of the brain affected by an ischemic stroke in a subject. The AID system 101 directs the user of a nerve stimulator therapeutic device to apply the nerve stimulation to the appropriate side of the head or body of the subject, eliminating need for bilateral stimulation. Other neurological conditions that may benefit from directed unilateral nerve stimulation include traumatic brain injury, migraine, seizure, and the like. In some embodiments, the AID system 101 can automatically deliver the therapeutic intervention through additional or integrated components to the subject 301 termed a therapy deliverer (not shown).

In another example, the AID system 101 determines whether the portion of the brain affected by the ischemic stroke is superficial/deeply located in the brain. A specific example of such anatomical localization is to diagnose injury to the cortex of the brain, versus injury to the subcortical structures such as the basal ganglia or thalamus. This distinction of injury site may determine specific treatments for the subject 301. The specific treatments include but may not be limited to endovascular recanalization/clot retrieval procedures.

In yet another example, the AID system 101 determines whether the brain affected by the ischemic stroke is located in forebrain, midbrain, or hindbrain. The AID system 101 may distinguish dysfunction localized in the telencephalon, diencephalon, mesencephalon, metencephalon, and/or myelencephalon. Said distinction may determine particular treatments for the subject 301. The particular treatments include but may not be limited to a nerve stimulator that is effective only at dilating arteries of the forebrain.

In some embodiments, the determination of the brain region affected by disease or other dysfunction is determined in part or in whole by the subject's symptoms and examination findings. Other embodiments may incorporate into the determination of disease-affect brain tissue various laboratory or neuroimaging test results.

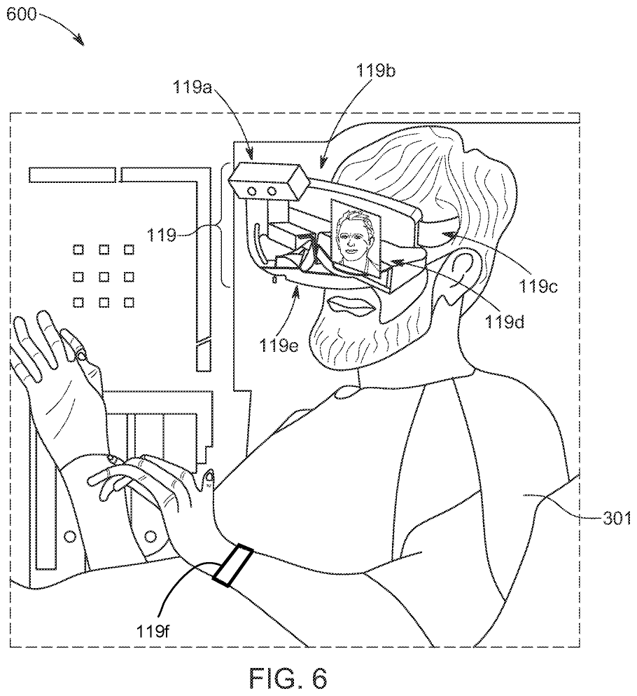

Referring to FIG. 6, a working example of a view 600 of the input/output device 119 associated with the AID system 101 is shown. Performance of the physical examination findings of the subject 301 by the AID system 101 preferably includes at least a minimum bidirectional verbal/audio communication, presentation of graphic or other visual information to the subject 301 or user of the AID system 101, and visualization of the subject's 301 face and/or body.

The view 600 includes the subject 301 and the input/output device 119. The subject 301 is the patient or any person who wants to interact with the AID system 101 for keeping a track of his/her medical health condition. In an embodiment, the input/output device 119 is a wearable device. The input/output device 119 is utilized by the subject 301 to interact with the AID system 101. The input/output device 119 displays graphic or other visual information to the subject 301 in response to the verbal interaction done by the subject 301 with the AID system 101. The input/output device 119 may be a portable device. In an example, the input/out device 119 is a headset. The subject 301 is wearing a headset. The headset includes one or more external cameras 119a facing toward the subject's body, one or more internal cameras 119b facing toward the subject's face and eyes, one or more speakers 119c, a semi-transparent augmented reality visor 119d, and one or more microphones 119e oriented at the subject's month or away from the subject. Each of the one or more external cameras 119a is a camera that is preferably capable of capturing caudal view hands and feet of the subject 301. In addition, each of the one or more internal cameras 119b is preferably capable of capturing close-up view of eyes and face of the subject 301. The one or more speakers 119c are preferably in proximity to ears of the subject 301. The one or more speakers 119c may be in direct contact with the head of the subject 301 if the subject 301 is suffering from conductive deafness.

A semi-transparent augmented reality visor 119d preferably shows an avatar image to the subject 301 and/or other information and images necessary for evaluation of the subject 301. Avatar image, a graphical representation of the subject, is created for the subject 301 by the AID system 101 in response to interaction with the subject 301. Avatar image is created to guide the subject 301, through the evaluation and to help the subject 301 understand accurately about the health condition diagnosed by the AID system or any recommendation of treatment provided by the AID system 101. In an example, the subject 301 may be an elderly person who cannot read. In such case, avatar image will help the subject 301 to understand the response of the AID system 101 well. The semi-transparent augmented reality visor 119d is capable of projecting graphical information to the subject 301 or for a user of the system while allowing the subject's view of the surrounding environment. Further, the microphone 119e is preferably attached to the headset 119 in direct proximity to mouth of the subject 301. The microphone 119e helps the subject 301 to interact with the AID system 101. Furthermore, the headset 119 preferably includes a plurality of externally facing microphones, externally facing speakers, and stimulators. Stimulators may be capable of delivering sensory stimulation to face and/or scalp of the subject 301. The sensory stimulation may be thermal, vibratory, tactile, or electrical in nature, and may deliberately increase in intensity to achieve or surpass a pain threshold. The headset 119 may include positional sensors. Positional sensors determine orientation of the headset in space. Positional sensors may encompass accelerometers, gyroscopes, and other sensors capable of determining position of the headset in space.

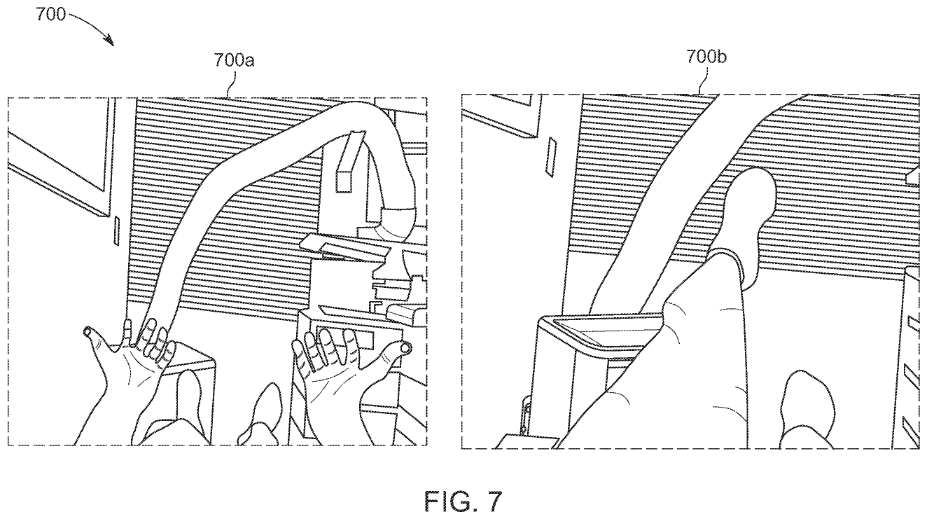

Referring to FIG. 7, visualization 700 of vantage points (700a, 700b) captured by the input/output device 119 associated with the AID system 101 is shown. The one or more external cameras 119a of the input/output device 119 may be utilized for visualization of arms (as shown in 700a) and visualization of legs (as shown in 700b). Visualization of arms and legs from the position where the one or more external cameras are placed, helps the AID system 101 to determine strength of arms and legs as a measure of absolute height of elevation and/or by comparing relative side-to-side elevation. Abnormal movements and coordination dysfunction may also be determined from these vantage points (700a, 700b).

The input/output device 119 may include wrist or ankle peripherals 119f connected with the headset wirelessly or through wires. The wrist or ankle peripherals 119f may be in the form of bracelets or adhesive pads. The wrist or ankle peripherals 119f may include position sensors to determine position of the extremity in space. Such sensors may encompass accelerometers, gyroscopes, and other sensors capable of determining position of a required component in space, and may include one or more batteries, processors, and/or memory modules. The wrist or ankle peripherals 119f may include stimulators capable of delivering sensory stimulation to the subject 301. Said stimulators may deliver electrical, thermal, movement, tactile, or other stimulation to the subject 301. Said stimulation may be intentionally made painful to the subject 301.

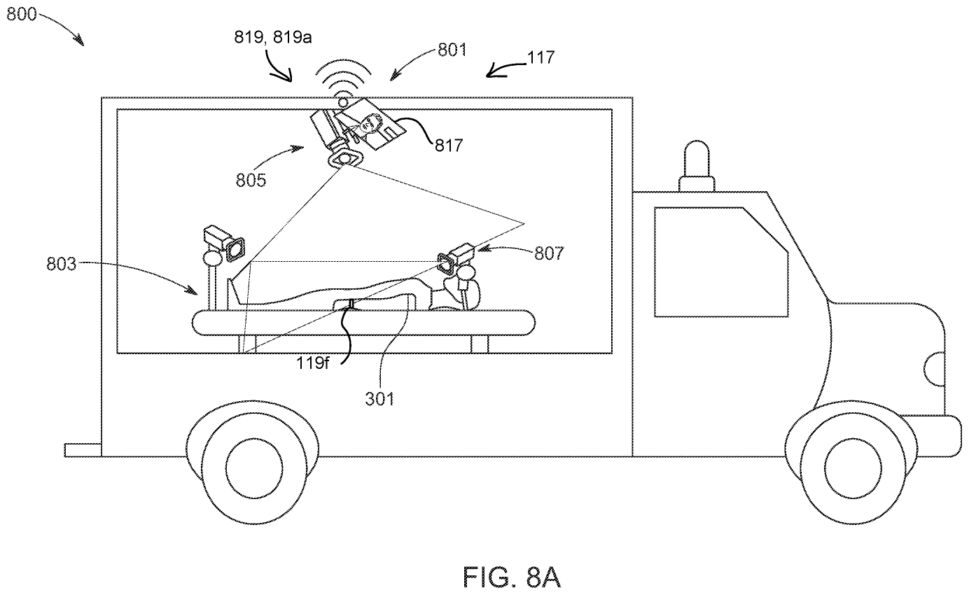

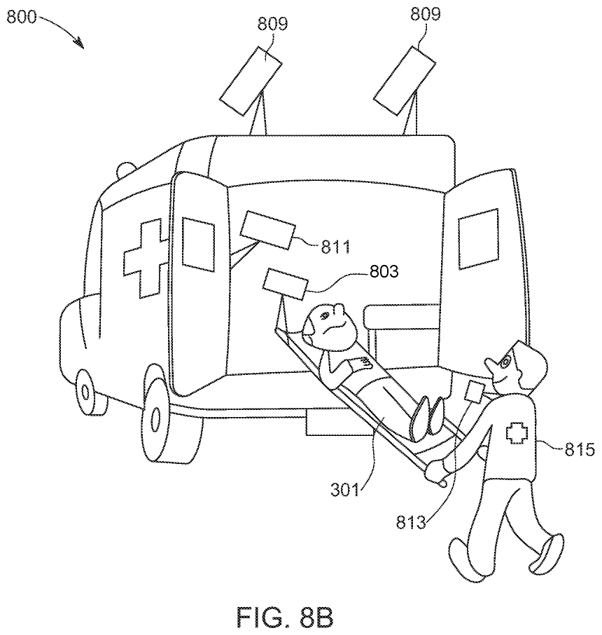

Referring to FIG. 8A, an ambulance 800 configured with the one or more sensors 117 associated with the AID system 101 is shown. The one or more sensors 117 may be a plurality of fixed equipment 801, 803 that are fixed to the ambulance 800. A first equipment 801 corresponds to a video camera 805, video console 817, speakers and microphone, Wi-Fi/cellular/Bluetooth transmitter/receiver 819 and/or signal booster 819a and the like. A second equipment 803 of the plurality of fixed equipment may be fixed to the gurney or stretcher, or other portable cart used to move a patient in and out of the ambulance vehicle. Other of fixed equipment may be fixed to the ambulance, a medic's clothing or supply pack, within the patient examination rooms in healthcare facilities such as hospitals and clinics, and/or in places where groups of people tend to congregate, or within the patient's home.

In the embodiment shown, the first equipment 801 includes a camera 805, and preferably a third-person camera, that may view the entire body, including the head, of the subject 301. By determining shapes, colors, and movements, such a camera could detect blood on the body of the subject 301, skin abnormalities such as rashes or burns, urine-soaked clothing, abnormal body postures, and limb or body movements. The third-person camera may work in conjunction with the one or more external cameras 119a of the headset to provide a complementary vantage point for evaluation of the subject 301.

first equipment 801 preferably includes a video console 817 capable of presenting the AID system's 101 avatar to the subject 301 and text readable by presbyopia subject. In general, presbyopia is the gradual loss of the eyes' ability to focus on nearby objects. It's a natural, often annoying part of aging. The first equipment 801 may also correspond to speakers and microphones to enable communication with the paramedic and other people in the ambulance 800. The first equipment 801 preferably has telecommunication capabilities 819, such as a wireless transmitter or other such device, and more preferably alternatively or additionally has a telecommunication signal amplification device 819a to improve cloud/internet connectivity. In addition, the first equipment 801 preferably has data processing and storage capabilities with a processor and memory module. The first equipment 801 preferably has storage and/or recharging dock ports for the headset and/or wrist or ankle peripherals.

The one or more sensors 117 may include a portable/wearable device 807. The portable/wearable device 807 may correspond to the input/output device 119. In some embodiments of the invention, various parts the input/output device 119 of the AID system 101 may be used to evaluate subjects with different conditions or in different situations. For example, as one form of a portable/wearable device 807, the headset 807 might offer limited diagnostic benefit in relation to communication with, or evaluation of, a comatose subject/patient, who by definition is unresponsive with closed eyes. The headset 807 might also offer limited diagnostic benefit in relation to communication with an agitated or combative subject/patient, whose behaviour could be exacerbated by application of the headset. However, the wrist and ankle peripherals 119f could be helpful in evaluation of the comatose patient, in whom response to pain is an important physical exam finding data element, but the wrist and ankle peripherals 119f might also not be helpful in evaluation of an agitated patient, in whom painful stimulation would only increase the patient's agitation. Peripherals 119f that are less restrictive, such as patches, may be better received by agitated patients, especially when not eliciting a pain response from the subject. The majority of patients with neurological emergencies are alert, attentive, and cooperative, and so would benefit from having all three parts (headset 807, wrist and/or ankle peripherals 119f, and one or a plurality of fixed equipment 801) of the input/output device 119 of the AID system 101 employed in their diagnostic evaluation. In this embodiment, when the headset 807 cannot be used by the patient, the patient evaluation will preferably be conducted through the fixed equipment 801 and/or peripherals 119f.

In some embodiments of the invention, the input/output device 119 of the AID system 101 comprises a processor 103, memory, 115, and instructions stored thereon, and other capabilities to run the AID system 101 and store any needed data for operation of the AID system 101 locally, on-site. Some embodiments may also utilize computational processes and services located remotely, and in further related embodiments can temporarily use on-site computational and data storage capabilities for certain functions or when telecommunications are limited.

Referring to FIG. 8B, a schematic diagram of internal and external view of the ambulance 800 configured with the one or more sensors 117 is illustrated. The one or more sensors 117 include one or more scene surveillance cameras 809. The one or more scene surveillance cameras 809 captures such vantage points that may be from external positions on the ambulance 800 surveying the surrounding environment to visualize and identify accident scenes. The one or more sensors 117 are preferably connected wired or wirelessly to the AID system 101.

The one or more sensors 117 may include a second equipment 803. The second equipment 803 may correspond to a camera installed on the gurney carrying the subject 301. The second equipment 803 allows subject's visualization during transport to and from the ambulance 800, and provides a different visual perspective to the first equipment camera 805, which aids in visual computation and evaluation and allows for improved visualization of a body part that may be partially or fully blocked from the first equipment camera 805. The one or more sensors 117 may include an additional internal camera 811 fixed inside the ambulance 800. The one or more sensors 117 may include a preferably smaller sized portable camera 813 connected to a paramedic 815. The one or more sensors 117 provide the collected information to the AID system 101 wirelessly or through wired data connection.

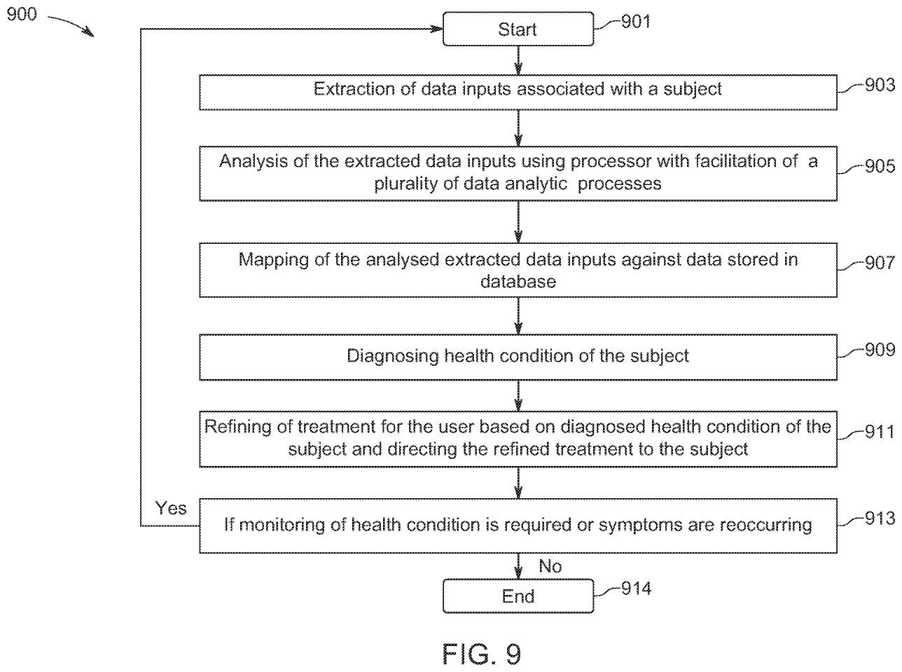

Referring to FIG. 9, a flow diagram of a method 900 for diagnosing health condition of the subject and directing refined treatment to the subject based on the diagnosed health condition is shown. The method 900 starts at step 901. Following step 901, at step 903, extraction of the data inputs associated with the subject is performed at the AID system 101. The AID system 101 preferably performs data extraction from the speech signal and the speech content of the subject. Additionally, data associated with physical examination findings may be extracted through computer vision analytics that assess various visually perceptible aspects of the physical condition of the subject. The various aspects of physical condition of the subject include but may not be limited to weakness in face or limbs of the subject, expressions on face of the subject, drooping eyelids, asymmetric pupils, deviation of the eye(s), tremor or jerking in the body, or unusual postures of the subject. The AID system 101 preferably extracts the data associated with visual physical examination findings through computer vision analytics using the one or more sensors 117. The one or more sensors 117 with facilitation of computer vision analytics captures one or more images focusing on abnormalities of the subject or subject-related images. The AID system 101 increases likelihood of accurate identification of normalities and abnormalities. A plurality of computer vision processing capabilities may be employed in a substantially parallel manner to examine a video or any visual representation of the subject. In some embodiments of the invention, the identification of normalities or abnormalities is a simple consensus or majority of the plurality of computer vision processing capabilities. Each of the computer vision processing capabilities may be considered equally capable of identifying normalities and abnormalities. In an exemplary embodiment of the invention, certain computer vision capabilities of the plurality of computer vision processing capabilities are preferentially selected or otherwise weighted to identify normalities and abnormalities contained in the one or more images of the based on the design, training, or accuracy of said computer vision capability. Data inputs also may include data from other sensors discussed above.

Accordingly, at step 905, analysis of the extracted data inputs is performed using the processor 103 with facilitation of the plurality of data analytics processes. The AID system 101 utilizes the plurality of data analytic process modules of the processor 103. The plurality of data analytic process modules of the processor 103 preferably includes two, three, or all four of MLP 105, SA 107, CM 109, and DCL 111 modules. Further, at step 907, mapping of the analyzed extracted data inputs against the data stored in the database 125 is performed, for example, by the CM module 109. At step 909, the health condition of the subject is diagnosed using the combined outputs of the plurality of data analytic process modules with facilitation of the diagnostic integrator 113. At step 911, refining of treatment for the subject is done based on the diagnosed health condition of the subject. In addition, the refined treatment is directed to the subject of the AID system 101. At step 913, the AID system 101 checks if monitoring of health condition of the subject is required or symptoms are reoccurring in the subject. If the subject or user chooses “yes” the evaluation of the subject by the AID system 101 may be performed iteratively and again start from the step 901. If the subject chooses “no”, the method terminates at step 914. Alternatively, the determination of the need to monitor the health condition of the subject may be determined according to internal criteria of the AID system, such as the severity of the patient's condition, the nature of the patient's diagnosis, the type of treatment recommended for the patient, and the duration of exposure of the AID system to the patient.

In some uses of the invention, the method 900 terminates at step 914. Alternatively, in some uses of the invention, the evaluation of the subject by the AID system 101 may be repetitious or iterative, repeating between every one and sixty minutes, or every hour, or between one and 12 times daily, for example. Additional evaluations of the subject by the AID system 101 may be desired to confirm, correct, or complement the information collected by previous evaluations, which then refines or revises the initial diagnosis and/or treatment regimen of the subject. Said additional evaluations of the subject by the AID system 101 may involve all or part of the typical processes of the AID system 101. Repeat evaluation of the subject by the AID system 101 may also be desired for monitoring the subject's condition for improvement (e.g., as a result of a treatment), deterioration (e.g., as the disease progresses), or recurrence, either during an initial encounter with the subject or over longer periods of time. It may be noted that the method 900 is explained to have above stated process steps; however, those skilled in the art would appreciate that the method 900 may have more or fewer process steps which may enable all the above stated embodiments of the present invention.

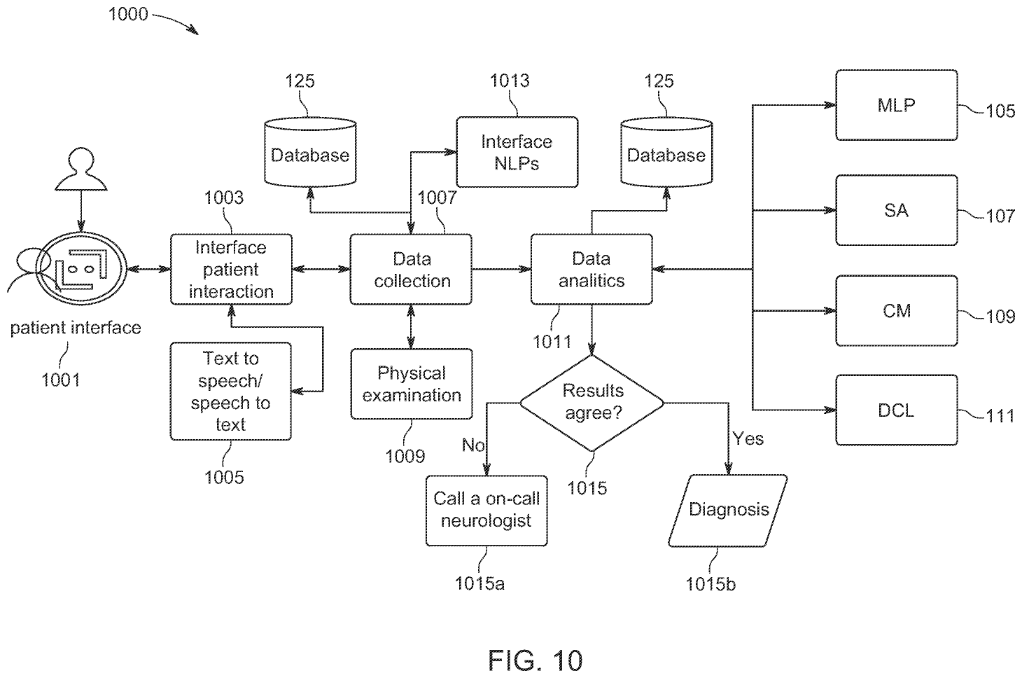

Referring to FIG. 10, a flowchart 1000 for functioning of an embodiment of the AID system 101 is illustrated. The functioning initiates at step 1001. At step 1001, patient interface is provided by the AID system 101. The patient interface may be in the form of an input/output device as described above 119. Any patient may interact with the AID system 101 using the patient interface. At step 1003, the patient interacts with the AID system 101 using the patient interface. At step 1005, the AID system 101 performs speech to text conversion or text to speech conversion based on requirement. For example, if the patient has interacted with the AID system 101 using speech or voice inputs, then the AID system 101 performs speech to text conversion to understand the patient's interaction accurately. At step 1007, the AID system 101 performs data collection or data extraction. The AID system 101 collects or extracts data inputs associated with the patient. The AID system 101 extracts or collects the data inputs through one or more of the patient, a physician, or any third-party/or third party platform. The data inputs extracted by the AID system 101 may correspond to clinical and non-clinical information collected by the physician or the third-party platform. In some embodiments, the data inputs may include data associated with medical history of the subject, family medical record, explanation of any health-related symptoms that the subject is having, medication use, allergies, physical examination findings, and basic laboratory testing results. At step 1009, the AID system 101 extracts data associated with physical examination findings through, for example, computer vision analytics using the one or more sensors 117 (as explained in FIG. 1). The one or more sensors 117 with facilitation of computer vision analytics capture one or more images focusing on abnormalities of the patient. The collected or extracted data/data inputs are stored in the database 125. The AID system 101 ensures accurate identification of normalities and abnormalities.

At step 1013, the collected data/data inputs or the converted speech to text data is analyzed to determine identity and/or nature of said data using a Natural Language Processing (NLP) interface.

At step 1011, the AID system 101 performs data analysis. The AID system 101 performs analysis of the extracted data inputs/data using the processor 103 with facilitation of the plurality of data analytics processes (as mentioned in FIG. 1). The AID system 101 utilizes the plurality of data analytic process modules of the processor 103. The plurality of data analytic process modules of the processor 103 includes two or more of MLP 105, SA 107, CM 109, and DCL 111 modules. The AID system 101 utilizes combination of two or more data analytic process modules of the plurality of data analytic process modules for data analysis. The MLP 105 analyzes the data inputs extracted from the patient or the third-party platforms (medical history and physical examination findings) by mapping to pre-established diagnosis present in the database 125. The extracted data/data inputs are evaluated by the SA 107 to identify matching classic syndromes, some of which may be associated with a serious health condition. Further, the AID system 101 performs mapping of the analyzed extracted data inputs with the data stored in the database 125 using the CM 109. In addition, the AID system 101 utilizes the DCL module 111 for generating one or more keywords and phrases as a part of diagnostic evaluation of the patient. The one or more keywords and phrases are linkable to healthcare service billing records. The healthcare service billing records contains a final diagnosis provided by treating physician. The healthcare service billing records may include the International Classification of Diseases or other such index as a means to standardize terminology and diagnosis. The one or more keywords and phrases may be used as indicators of an individual subject's/patient's diagnosis during that subject's/patient's evaluation. The measure of certainty of a keyword/phrase linked to a diagnosis may be numerical, proportional, based upon frequency of occurrence, determined by specificity, and/or involve some other measure of the quality or strength of the link between the one or more keywords and phrases and the diagnostic code.

At step 1015, the AID system 101 provides results of diagnosis from each of the plurality of data analytics processes. At step 1015a, the AID system 101 calls an on-call neurologist, for example, or other appropriate physician for further assistance for the patient if predetermined criteria for agreement between the plurality of data analytic process modules are not metadata analytic process modules. The physician contacted is preferably a physician whose specialty training is related to the (certain) diagnosis or uncertain diagnosis of the patient. At step 1015b, the AID system 101 provides the diagnosis to the patient and/or healthcare provider user(s) if both of the two data analytic process modules of the plurality of data analytic process modules agree on the diagnosis of stroke or any health condition.

In further embodiments, the disclosed invention improves the identification of medical terminology provided to the AID system 101 by the subject/patient in the form of natural speech utterances wherein the medical terminology may be obscured deliberately or unintentionally by the subject/patient as: polysemous, ambiguous, equivocal, or vague word choices; amphibolic sentence structures; analogies; or slang.

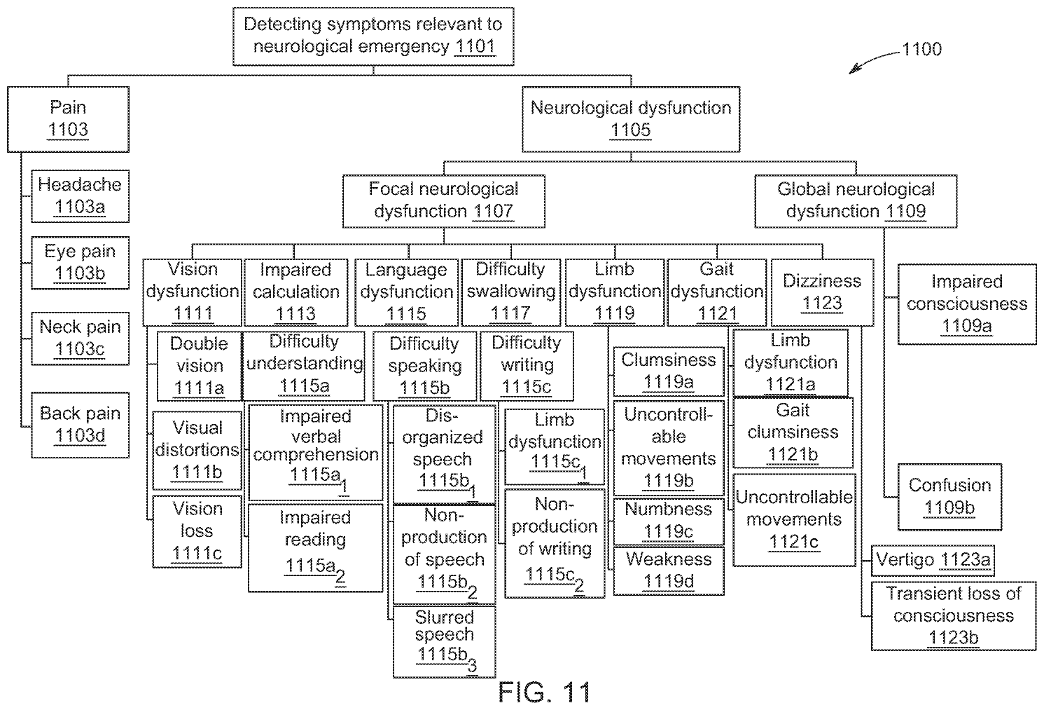

In one such embodiment, medical terminology is structured as a hierarchy within the AID system 101 within which an utterance made by the patient triggers one or more specific subheadings of the hierarchy. The various subheadings in the hierarchy identified in this manner thereby indicate or identify the medical term at a higher level in the hierarchy (e.g., a categorical term) that best represents the subject/patient's utterance, and the categorical term in the medical hierarchy is subsequently used by the AID system 101 as a data input for the diagnostic process(es). Referring to FIG. 11, for example, a hierarchy 1100 for detecting symptoms relevant to a neurological emergency is illustrated. The medical terminology hierarchy employed by the AID system 101 encompasses symptoms relevant to the neurological emergency, detected at step 1101. The detected symptoms are then subdivided into symptoms of Pain 1103 and symptoms of Neurological Dysfunction 1105 as categorical terms. The categorical term Pain 1103 is further divided into: headache 1103a, eye pain 1103b, neck pain 1103c, and back pain 1103d. The categorical term Neurological Dysfunction 1105 is further divided into symptoms of Focal Neurological Dysfunction 1107 and symptoms of Global Neurological Dysfunction 1109, which are also categorical terms. The categorical term Focal Neurological Dysfunction 1107 is further subdivided into symptoms of vision dysfunction 1111, impaired calculation 1113, language dysfunction 1115, difficulty swallowing 1117, limb dysfunction 1119, gait dysfunction 1121, and dizziness 1123. Vision dysfunction 1111 has following symptoms: double vision 1111a, visual distortions 1111b, and vision loss 1111c. Language dysfunction 1115 is further divided into 3 terms: difficulty understanding 1115a, difficulty speaking 1115b, and difficulty writing 1115c. Difficulty understanding 1115a has following symptoms: impaired verbal comprehension 1115a₁, and impaired reading 1115a₂. Difficulty speaking 1115b may have the following symptoms: disorganized speech 1115b₁, non-production of speech 1115b₂, and slurred speech 1115b₃. Difficulty writing 1115c may have the following symptoms: limb dysfunction 1115c₁, and non-production of writing 1115c₂. Limb dysfunction 1119 may have the following symptoms: clumsiness 1119a, uncontrollable movements 1119b, numbness 1119c and weakness 1119d. Further, gait dysfunction 1121 may have the following symptoms: limb dysfunction 1121a, gait clumsiness 1121b, and uncontrollable movements 1121c. Dizziness 1123 may have the following symptoms: vertigo 1123a, and transient loss of consciousness 1123b. The categorical term Global Neurological Dysfunction 1109 may have the following symptoms: impaired consciousness 1109a and confusion 1109b.

In an example, a patient who is subject to evaluation by the AID system 101 reports that he has experienced 3 symptoms: “vision loss 1111c”, “slurred speech 1115b₃”, and “weakness 1119d”. The 3 symptoms described by the patient/subject are represented by 3 subheading terms in the hierarchy, all of which are within the domain of the categorical term Focal Neurological Dysfunction 1107. Since Focal Neurological Dysfunction 1107 may be caused by medical conditions such as stroke, the evaluation of the subject/patient then immediately proceeds to additional steps intending to diagnose the patient with stroke in preference of other evaluations.

A subject or a patient whose utterances relate to multiple subheading terms in the medical hierarchy that are not all contained within a single categorical term cannot be presumed to have a certain medical diagnosis related to a categorical term, and thus the evaluation of said subject/patient could not be specifically directed toward identification of that certain medical diagnosis to the exclusion of other evaluations. In such a counterexample, the subject/patient may provide an utterance for evaluation to the AID system 101 in which the specific terms “back pain 1103d”, “slurred speech 1115b₃”, and “confusion 1109b” are recognized. Each of the three recognized specific terms are subheadings contained within distinct categorical terms, preventing any assumptions about the disease condition as being related to one specific categorical term.

In other embodiments, an utterance made by the subject/patient indicates or otherwise is relatable to a category within a hierarchy of medical terms wherein the categorical term is not suitably precise to serve as a data input for the diagnostic process(es) of the AID system 101, but wherein the imprecise category contains within it several precise medical terms that individually would serve as data inputs for the diagnostic process(es). In order to determine which of the precise medical terms contained within the imprecise category are relevant to the subject/patient's utterances describing symptoms, a subroutine within the AID system 101 is thereby activated for the purpose of distinguishing the application of the various precise medical terms contained in the imprecise category to the subject/patient's utterances.

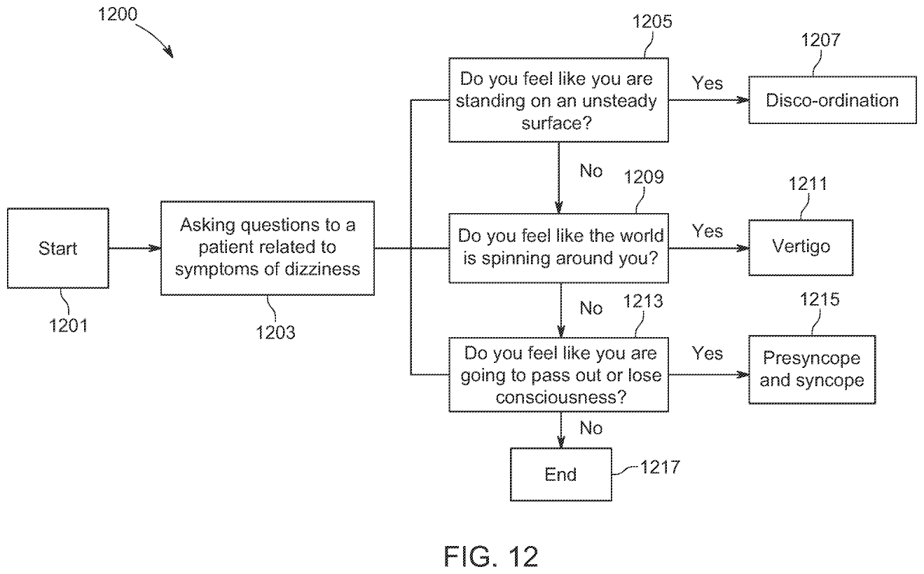

The subroutine of the AID system 101 intended to distinguish between a plurality of precise medical terms contained within an imprecise category may take several forms and be dependent upon the nature of the imprecise category. Referring to FIG. 12, a use case with a process flowchart 1200 for asking questions to a patient related to symptoms of dizziness is illustrated. In one embodiment of such a subroutine, shown in FIG. 12, the patient's utterance is only sufficient to satisfy the requirements for the imprecise categorical term “dizziness”, which labels a category of precise terms that includes “discoordination”, “vertigo”, and “presyncope and syncope”. Within the imprecise category of “dizziness”, all three precise terms are considered as equally probable interpretations of a patient's utterance referencing dizziness. The subroutine intended to distinguish between the precise terms within the imprecise category of “dizziness” then asks a set of predetermined questions to identify which one or more of the precise terms is an appropriate description of the subject/patient's experience, i.e., symptoms. The precise term(s) determined to be appropriate for the subject/patient's utterance then are usable as data inputs for the diagnostic process(es) of the AID system 101.

At step 1201, the process flowchart 1200 starts. At step 1203, the set of predetermined questions are asked to a subject/patient related to symptoms of the imprecise symptom, such as dizziness in this example. At step 1205, the AID system 101 is configured to generate an output corresponding to asking of a first of multiple questions to more precisely identify the symptom, here being, “Do you feel like you are standing on an unsteady surface?” to the patient. If the patient says “yes”, then at step 1207, discoordination is identified in the patient. Regardless of the subject/patient's answer being affirmative or negative at step 1207, the process advances to step 1209. At step 1209, the AID system 101 is configured to generate an output corresponding to a second question being asked from the patient. The second question may be, “Do you feel like the world is spinning around you?” If the patient says “yes”, then at step 1211, vertigo is identified in the patient. Regardless of the subject/patient's answer at step 1209, the process advances to step 1213. At step 1213, the AID system 101 is configured to generate an output corresponding to a third question being asked from the patient. The third question may be, “Do you feel like you are going to pass out or lose consciousness?” If the patient says “yes”, then at step 1215, presyncope and syncope is identified in the patient. Then the process flowchart 1200 ends after answers to all 3 questions are received by the AID system regardless of the affirmative or negative nature of the answers. In some embodiments, the process flowchart 1200 may end if any of the 3 questions receives a predetermined answer from the subject/patient. The questions asked are not limited to the above listed questions.

In another embodiment of the subroutine of the AID system 101 is intended to distinguish between a plurality of precise medical terms contained in an imprecise category, the precise terms are not equally probable descriptions of the subject/patient's utterance and/or in certain instances may be exclusive. The unequal probability of the precise medical terms contained in the imprecise category may be predetermined by the AID system based on the frequency of previous patient evaluations, medical literature data, expert opinion, or other sources of information, or else the unequal probability of the precise medical terms contained in the imprecise category may be determined during the evaluation of the subject/patient as a result of other information known to or obtained by the AID system about the subject/patient.

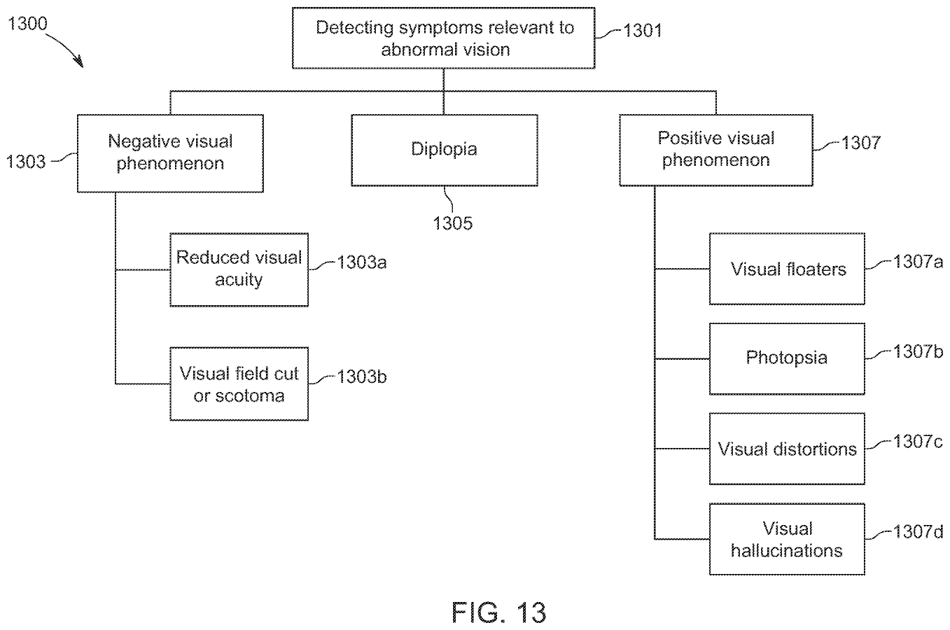

Referring to FIG. 13, a term hierarchy for a use case of a workflow 1300 for detecting symptoms relevant to abnormal vision is illustrated. In this example, at step 1301, symptoms relevant to abnormal vision are detected from the utterances made by a subject/patient. The symptoms relevant to abnormal vision are categorized into 2 categorical terms (Negative Visual Phenomenon 1303, and Positive Visual Phenomenon 1307) and a single precise term (diplopia 1305) The categorical term Negative Visual Phenomenon 1303 includes the precise terms ‘reduced visual acuity’ 1303a and ‘visual field cut or scotoma’ 1303b. The categorical term Positive Visual Phenomenon 1307 includes precise terms ‘visual floaters’ 1307a, ‘photopsia’ 1307b, ‘visual distortions’ 1307c, and ‘visual hallucinations’ 1307d. However, not all of the terms are equally probable descriptions of the subject/patient's utterances: ‘diplopia’ 1305, ‘visual field cut or scotoma’ 1303b, and ‘reduced visual acuity’ 1303a are more commonly representative of patient utterances than are the other terms. To note, each of the precise terms, or symptoms, 1303a, 1303b, 1307a, 1307b, 1307c, and 1307d are data elements, of the type utilized by the SA 107.

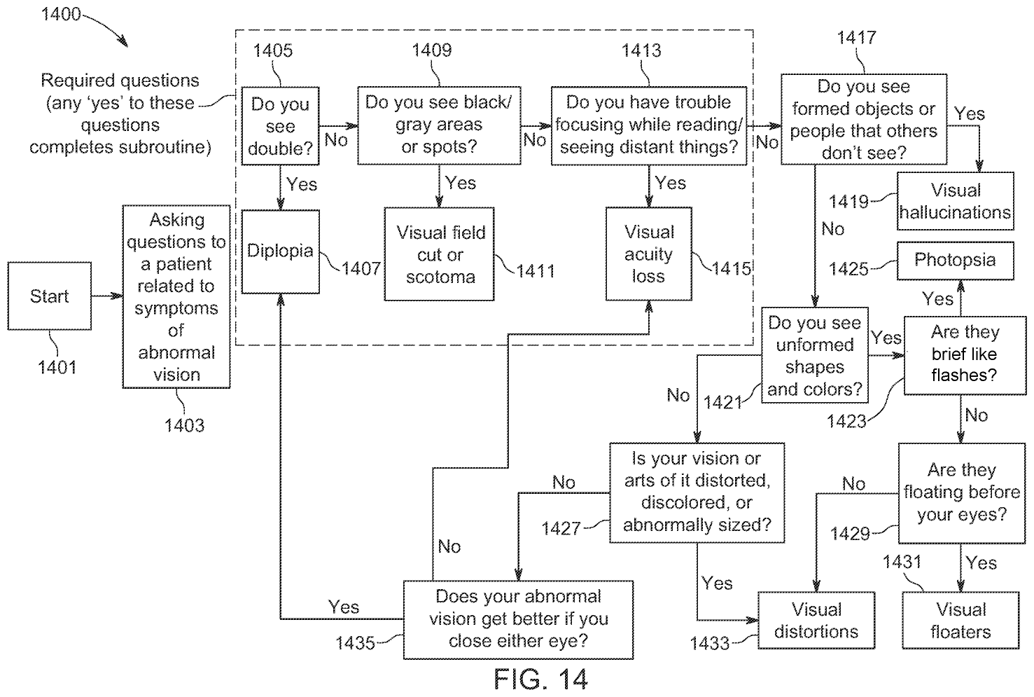

Referring to FIG. 14, then, a use case with a flowchart 1400 for asking questions to a patient related to symptoms of abnormal vision is illustrated. The subroutine designed to distinguishing between the precise term(s) gives preference to the commonly representative precise terms within the imprecise category, e.g., as the flowchart 1400 of questions (FIG. 14) that initially queries the subject/patient to confirm the appropriateness of the commonly-representative precise terms to the subject/patient's nebulous utterance. The subroutine completes its function if it confirms the appropriateness of one or more of the commonly-representative precise terms to the subject/patient's utterance, and it would only consider the appropriateness of other, less-commonly invoked precise terms contained within the imprecise category if no attribution of the commonly-representative precise terms can be made to the subject/patient's utterances.

The flowchart initiates at step 1401. At step 1403, a set of questions related to symptoms of abnormal vision are asked to a patient. At step 1405, a first question, “Do you see double?” is asked by the AID system 101 to the patient. If the patient says “yes”, diplopia 1407 is identified in the patient. Regardless of the subject/patient's answer in step 1405, step 1409 is then followed. At step 1409, a second question, “Do you see black/gray areas or spots?” is asked. If the patient says “yes”, visual field cut or scotoma 1411 is identified in the patient. Regardless of the subject/patient's answer in step 1409, step 1413 is followed. At step 1413, a third question, “Do you have trouble focusing while reading or seeing distant things?” is asked. If the patient says “yes”, visual acuity loss 1415 is identified in the patient. Any “yes” or otherwise affirmative answer to the questions asked at step 1405, 1409 and 1413 completes the subroutine as these 3 questions all must be asked of any subject/patient who has abnormal vision, and more than one of the 3 precise terms queried by those questions may apply to the subject/patient's utterance. However, if no affirmative response is obtained to any of the 3 required questions of the subroutine, step 1417 is followed.

At step 1417, a fourth question, “Do you see formed objects or people that others don't see?” is asked. If the patient says “yes” at step 1417, visual hallucinations 1419 are identified in the patient and the subroutine ends. If the patient says “no” at step 1417, then step 1421 is followed. At step 1421, a fifth question, “Do you see unformed shapes and colors?” is asked. If the patient says “yes” at step 1421, step 1423 is followed. At step 1423, a sixth question, “Are they brief like flashes?” is asked. If the patient says “yes” at step 1423, photopsia 1425 is identified in the patient and the subroutine ends. If the patient says “no” at step 1423, step 1429 is followed. At step 1429, a seventh question is asked, “Are they floating before your eyes?”. If the patient says “yes” at step 1429, visual floaters 1431 are identified in the patient and the subroutine ends. If the patient says “no” at step 1429, then visual distortions 1433 are identified in the patient and the subroutine ends. If the patient says “no” to the fifth question, steps 1423 and 1429 are skipped and step 1427 is followed. At step 1427, an eighth question is asked, “Is your vision or parts of it distorted, discolored, or abnormally sized?”. If the patient says “yes” at step 1427, visual distortions 1433 are identified in the patient and the subroutine ends. If the patient says “no”, step 1435 is followed. At step 1435, a ninth question is asked, “Does your abnormal vision get better if you close either eye?”. If the patient says “yes” at step 1435, diplopia 1407 is identified in the patient and the subroutine ends. If the patient says “no”, visual acuity loss 1415 is identified in the patient and the subroutine ends. The questions asked may not be limited to the mentioned questions.

Many modifications and other embodiments of the inventions set forth herein will come to mind to one skilled in the art to which these inventions pertain having the benefit of the teachings presented in the foregoing descriptions and the associated drawings. It is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1-20. (canceled)

21. A medical device comprising:
a processor comprising a plurality of data analytic process modules and a diagnostic integrator;
a memory communicably coupled to the processor;
an input/output device communicably coupled to the processor,
the processor being configured to execute instructions stored in the memory to:
cause the patent interface to record a first data from a subject;
analyze the first data with a first of the plurality of data analytic process modules and determine a first diagnostic output;
analyze the first date with a second of the plurality of data analytic process modules and determine a second diagnostic output;
integrate the diagnostic outputs from the plurality of data analytic process modules and determine a unified final diagnosis to a subject.

22. The medical device of claim 21, wherein the input/output device includes at least one sensor.

23. The medical device of claim 22, wherein the at least one sensor includes a video camera and a microphone.

24. The medical device of claim 22, wherein the at least one sensor further include one or more of and thermal camera, thermometer, electrocardiography sensor, photoplethysmography sensor, electromagnetic pulse monitor, accelerometer, and a gyroscope.

25. The medical device of claim 21, wherein the input/output device includes one or more of a speaker and video display screen.

26. The medical device of claim 21, wherein the input/output device comprises a headset wearable by the subject.

27. The medical device of claim 26, wherein the headset comprises one or more external cameras facing in a direction not towards a face of the subject when the subject is wearing the headset, one or more internal cameras facing toward the face of the subject when the subject is wearing the headset, one or more speakers, a semi-transparent augmented reality visor, and one or more microphones oriented proximate to a mouth of the of the subject when the subject is wearing the headset, one or more speakers oriented proximate to ears of the subject.

28. The medical device of claim 21, wherein the input/output device comprises one or more stimulators positioned to deliver sensory stimulation to face, scalp, and/or other body part of the subject, wherein the stimulation delivered is on or more of thermal, vibratory, tactile, and/or electrical in nature.

29. The medical device of claim 21, wherein the input/output device comprises one or more peripherals positioned on one or both ankles and/or one or both wrists of the subject, the peripherals including adhesive and or having a circular shape to remain frictionally attached to a subject wrapped around a limb of the patient, the peripherals including one or more sensors and or one or more stimulators.

30. The medical device of claim 21, further comprising a plurality of fixed equipment, wherein the each of the plurality fixed equipment is fixed to a respective one of a vehicle, a building, a medical transport, and a furniture.

31. The medical device of claim 30, wherein a first equipment of the plurality of fixed equipment is fixed to an ambulance and includes a third person video camera, a video console, one or more speakers, and a microphone.

32. The medical device of claim 31, wherein a second equipment of the plurality of fixed equipment is fixed to a medical transport used to move a patient in and out of the ambulance vehicle.

33. The medical device of claim 21, wherein the processor is further configured to cause the input/output device to display graphic and/or other visual information to the subject in response to verbal response received from subject to auditory, the subject verbal response being in response to visual or auditory output from the medical device.

34. The medical device of claim 21, wherein the plurality of data analytic process modules includes at least two of includes a machine learning process module, a syndrome analyzer module, a case matching module, and a diagnostic code linking module.

35. The medical device of claim 21, wherein the processor is further configured to convert patient speech to text and cause speakers to auditorily respond to patient with spoken text.

36. The medical device of claim 21, wherein the processor is further configured to access one or more databases.

37. The medical device of claim 34, wherein the machine learning process module determines likelihood of proper diagnosis of a given disease or condition in the subject based on a combined association of a plurality of data inputs and the incidence of given disease or condition, where the data inputs are collected from the subject through the input/output device, and data inputs include one or more of presence of sudden numbness or weakness in body of the subject, a National Institutes of Health Stroke Scale (NIHSS) score, indication of tobacco, an age, a race, a sex, indication of dyslipidemia, indication of atrial fibrillation, indication of high blood pressure, current systolic blood pressure, current diastolic blood pressure, current glucose level, medications the subject is currently taking, indication of subject family history of stroke, indication of coronary artery disease, and current heart rate.

38. The medical device of claim 34, wherein the syndrome analyzer module determines likelihood of proper diagnosis of a given disease or condition in the subject based on a presence or absence of one or more data elements, where data elements are symptoms associated with the disease or condition.

39. The medical device of claim 21, further comprising a therapy deliverer, wherein, after the processor determines a diagnosis of a disease, the processor is further configured to cause the therapy deliverer to deliver a therapy directly to the subject.

40. The medical device of claim 39, wherein the therapy deliverer delivers one of injection of medication and electrical nerve stimulation to the subject.
